(19) United States
(12) Patent Application Publication
(10) Pub. No.: US 2023/0091535 A1
(43) Pub. Date: Mar. 23, 2023

(54) COMPOSITIONS OF FREE FLOWING HYDROPHOBIC CARBOHYDRATE DERIVATIVES AND METHODS FOR USE

(71) Applicant: CORN PRODUCTS DEVELOPMENT, INC., Westchester, IL (US)

(72) Inventors: Maryvonne FUENTES, Bridgewater, NJ (US); Ashraf HOSSAIN, Bridgewater, NJ (US); Christina ODORISIO, Bridgewater, NJ (US); Anna YACHERA, Bridgewater, NJ (US)

(21) Appl. No.: 17/797,472

(22) PCT Filed: Jan. 19, 2021
(86) PCT No.: PCT/US2021/013867

§ 371 Date: Aug. 4, 2022

Related U.S. Application Data
(60) Provisional application No. 62/975,532, filed on Feb. 12, 2020.

(51) Int. Cl. A61K 8/73; A61K 8/19; A61K 8/27; A61K 8/29

(52) U.S. Cl. CPC A61K 8/732; A61K 8/19; A61K 8/27; A61K 8/29; A61Q 1/12

(57) ABSTRACT

Disclosed herein is a free flowing, oil absorbing and water repellant modified carbohydrate, wherein the modified car-bohydrate is produced from a carbohydrate base being derivatized with one or more anionic moieties; esterified with octenylsuccinic anhydride; and complexed with a polyvalent cation, as well as compositions containing such modified carbohydrates. Such modified carbohydrates and compositions containing same are used in food and personal care applications, cosmetics, dry shampoo, anti-perspirant, deodorant, ointment and sunscreen.

### CROSS-REFERENCE TO RELATED APPLICATIONS

This application is related to and claims the benefit of priority from U.S. Provisional Patent Application Ser. No. 62/975,532, filed Feb. 12, 2020, which is hereby incorporated herein by reference in its entirety.

### BACKGROUND

Disclosed herein are octenylsuccinate modified carbohydrate derivative salts, useful in commercial applications, such as food ingredients, industrial ingredients, cosmetics, dry shampoo, anti-perspirant, deodorant, ointment and sunscreen, and compositions containing same. Further disclosed herein are methods of making and using the octenylsuccinate modified carbohydrate derivative salts and compositions containing such modified carbohydrates.

Over the years, starch octenylsuccinate derivatives have been used in a variety of industrial, food, pharmaceutical and cosmetic applications, for their ability to stabilize emulsions. Aluminum starch octenylsuccinate, a hydrophobic starch with good free flow and water repellant properties, has been used in a number of different applications. Exemplary disclosures showing aluminum starch octenylsuccinates can be found in U.S. Pat. No. 5,013,763, where the starch modified aluminum salt was used in skin preparations such as hand or body lotions, and in U.S. Pat. No. 5,407,678, where it was used in petroleum jelly cosmetic compositions. Further disclosures of the aluminum salt of starch modified octenylsuccinate are seen in U.S. Pat. No. 3,852,475, where it was found in topical compositions containing petrolatum; in U.S. Pat. No. 4,994,264 in press molded cosmetic compositions; and in U.S. Pat. No. 4,894,222 where it was found in sunscreen preparations.

While aluminum starch octenylsuccinate is a hydrophobic starch with good free flow and water repellant properties, recent health and ecological concerns raised health about the use of aluminum metals in personal care and food applications has inspired efforts to find a replacement or substitute hydrophobic starch that has good free flow and water repellant properties. Starches modified with octenylsuccinate anhydride (OSA) and its salts, particularly calcium, however, have been found deficient in one or more of these desired properties (e.g. free flow and hydrophobicity).

See, e.g., U.S. Pat. No. 5,776,476. In view of the foregoing, there is a need to provide a hydrophobic carbohydrate that exhibits both good free flow and good water repellant properties, and provides those properties in industrial, food, pharmaceutical and cosmetic formulations.

### BRIEF DESCRIPTION OF THE DRAWINGS

FIG. 1 shows the water repellency properties of a sunscreen formulation made with octenylsuccinate modified carbohydrate derivative salt.

### DETAILED DESCRIPTION

One aspect is directed to a free flowing, oil absorbing and water repellant modified carbohydrate, wherein the modified carbohydrate is produced from a base carbohydrate derivatized with one or more anionic moieties; esterified with an alkyl or alkenyl succinic anhydride; and complexed with a polyvalent cation. Another aspect is directed a composition comprising a free flowing, oil absorbing and water repellant modified carbohydrate, wherein the modified carbohydrate is a carbohydrate base derivatized with one or more anionic moieties; esterified with an alkyl or alkenyl succinic anhydride; and complexed with a polyvalent cation.

In yet another aspect, the one or more anionic moieties is a carboxylate, sulfonate, phosphate, or combination thereof. In still other aspects, the carbohydrate base is a starch, cellulose, gum, or mixture thereof In yet further aspects, the carbohydrate base is starch sourced from corn, high amylose corn, waxy corn, potato, pea, rice, waxy rice, sago, tapioca, waxy tapioca, or mixtures thereof In still other aspects, the alkyl or alkenyl succinic anhydride of any of the preceding aspects is octenylsuccinic anhydride. In even still further aspects, the polyvalent cation of any of the preceding aspects is a metal ion and/or alkaline earth metal ion. In another aspect, the polyvalent cation is calcium, zinc, iron, copper, titanium or a mixture thereof In still other aspects, the angle of repose of the composition of any one of the preceding aspects is between about 20 to about 35. In still even further aspects, the carbohydrate base is starch, functional anionic moiety is a carboxylate, the alkyl or alkenyl succinic anhydride is octenylsuccinic anhydride, and the polyvalent cation is calcium.

In further aspects, the composition of any of the preceding aspects is a personal care composition. In yet further aspects, the personal care composition is a cosmetic, dry shampoo, anti-perspirant, deodorant, body powder, ointment or sunscreen. In certain aspects, the composition of any of the preceding aspects is a food composition. In still other aspects, the composition of any of the preceding aspects is an industrial composition.

Some other aspects are directed to a method of using the composition of any of the preceding aspects in a food, cosmetic, dry shampoo, anti-perspirant, deodorant, ointment, sunscreen, or industrial application. Still further aspects are directed to use of the composition of any one of the preceding aspects in a food, cosmetic, dry shampoo, anti-perspirant, deodorant, ointment, sunscreen, or industrial application.

Other aspects are directed to a method of preparing a free flowing, oil absorbing and water repellant modified carbohydrate, wherein the modified carbohydrate is provided by a carbohydrate base derivatized with one or more anionic moieties; esterified with an alkyl or alkenyl succinic anhydride; and complexed with a polyvalent cation, wherein said method comprises: i) treating a carbohydrate base to produce a carbohydrate derivative; ii) esterifying the carbohydrate derivative with alkyl or alkenyl succinic anhydride to produce a carbohydrate derivative esterified with an alkyl or alkenyl succinate; and iii) mixing the carbohydrate derivative esterified with alkyl or alkenyl succinate with a polyvalent cation. In some aspects, the method of any of the preceding aspects uses calcium as the polyvalent cation. In other aspects, the method of any of the preceding aspects uses a modified carbohydrate esterified with alkyl or alkenyl succinate that is, prior to mixing with the polyvalent cation, at pH of about 8. In still other aspects, the source of calcium used in the method of any of the preceding aspects is calcium acetate, calcium chloride, or a mixture thereof.

In yet other aspects, the method of any of the preceding aspects uses sodium hypochlorite at a level of 0.1 to 17%, 0.1 to 15%, 0.1 to 13%, 0.1 to 10%, 0.1 to 5%, 0.1 to 3%, 0.1 to 1%, or 0.4-1.2% active chlorine based on weight of carbohydrate to treat the carbohydrate base to produce a carbohydrate derivative. In certain aspects, the esterifying step within the method of any of the preceding aspects further uses octenylsuccinic anhydride as the alkyl or alkenyl succinate anhydride. In certain aspects, the esterifying step used in the method of any of the preceding aspects further uses about 1-5% octenylsuccinic anhydride based on the weight of carbohydrate. In certain aspects, the mixing step of any of the preceding aspects uses calcium at 4-20 times the stoichiometric equivalent to the anionic functional groups present on the carbohydrate.

The invention will now be described with reference to various exemplary embodiments and/or aspects. It is to be understood that the embodiments/aspects of the invention described herein are only intended to enable those skilled in the art to better understand and further implement the invention, and not intended to limit the scope of the invention disclosed herein in any manner. One skilled in the art will readily recognize from the following description that alternative embodiments of the structures and methods illustrated herein may be employed without departing from the principles of the invention disclosed herein. Modifications and variations of the invention disclosed herein can be made without departing from its spirit and scope, as will be apparent to those skilled in the art. Functionally equivalent methods to those described herein, in addition to those enumerated herein, will be apparent to those skilled in the art. Such modifications and variations are intended to fall within the scope of the appended claims. It is to be understood that the embodiments and aspects described herein are not limited to methods, conjugates, reagents, compounds, or compositions, which can, of course, vary. All methods described herein can be performed in any suitable order unless otherwise indicated herein or otherwise clearly contradicted by context.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention. Additionally, the phrase “consisting essentially of will be understood to include those elements specifically recited and those additional elements that do not materially affect the basic and novel characteristics of the claimed technology. The phrase “consisting of excludes any element not specified.

As will be understood by one skilled in the art, for any and all purposes, particularly in terms of providing a written description, all ranges disclosed herein also encompass any and all possible subranges and combinations of subranges thereof. As used throughout, ranges are used as shorthand for describing each and every value that is within the range. Any value within the range may be selected as the terminus of the range. All language such as “up to,” “at least,” “greater than,” “less than,” and the like, include the number recited and refer to ranges which can be subsequently broken down into subranges as discussed above.

Use of “about” to modify a number in this specification is meant to include the number recited plus or minus 10%. Where legally permissible recitation of a value in a claim means about the value. Use of about in a claim or in the specification is not intended to limit the full scope of covered equivalents.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure. In the event of a conflict between a definition in the present disclosure and that of a cited reference, the present disclosure controls.

Disclosed herein is a free flowing, oil absorbing and water repellant modified carbohydrate, wherein the modified carbohydrate is a base carbohydrate derivatized with one or more anionic moieties; esterified with an alkyl or alkenyl succinic anhydride; and complexed with a polyvalent cation. Still further disclosed herein are alkyl or alkenyl succinate modified carbohydrate derivative salts. Even further disclosed herein are octenylsuccinate modified carbohydrate derivative salts.

Still even further disclosed herein are compositions comprising one or more modified carbohydrate disclosed herein. Compositions comprising such modified carbohydrate surprisingly exhibit beneficial free flowing, oil absorbing and water repellant properties. Such compositions are useful as emulsion stabilizers in various commercial applications for use including, but not limited to, for example, food or industrial compositions or ingredients, cosmetics, dry shampoo, conditioner, anti-perspirant, deodorant, ointment and sunscreen. In certain aspects, the compositions are useful in rich creams and lotions. In certain aspects, the compositions are useful in sun care products, dry shampoos, conditioners, deodorants and anti-perspirants, and make-up.

Some embodiments are directed to a composition comprising a free flowing, oil absorbing and water repellant modified carbohydrate, wherein the modified carbohydrate is produced from a carbohydrate base being derivatized with one or more anionic moieties; esterified with an alkyl or alkenyl succinate; and complexed with a polyvalent cation (where esterified with octenylsuccinic anhydride, also referred to herein as “octenylsuccinate modified carbohydrate derivative salt”). Methods to determine free flow, oil absorption and water repellant properties are provided herein.

The purity of the composition may vary depending on the application needs. In certain embodiments, the composition is made only of the modified carbohydrate. In such embodiments, the composition is 100% pure modified starch. In other embodiments, the composition is about 99%, 90-99, 85-99, 80-99, 70-90, 50-70, 30-70, 10-30, 5-10, or 0.1-5% pure modified starch.

In some embodiments, the carbohydrate base material may be derived or obtained from any carbohydrate source. In other embodiments, the carbohydrate base is a starch, cellulose, gum, or mixture thereof.

Such carbohydrate source includes, but is not limited to, starches derived from any plant source including from cereals like sorghum, corn, wheat, barley, oats, triticale and rice; tubers like potato and tapioca; legumes such as pea and lentil; piths of various palm stems like sago; waxy starches such as waxy maize, waxy cassava, waxy potato and waxy rice; tapioca, and high amylose starch such as high amylose corn, i.e., starch having at least 40%, and more particularly, at least 65% amylose content by weight, and others including starches derived from conventional inbred breeding techniques or from genetically modified plant species. In certain aspects, starch flour may be used.

In certain embodiments, the carbohydrate base is purified from the carbohydrate source to about 70%, 80%, 90%, or preferably 95% or 99% purity. Purification is performed using methods known to one of skill in the art. In other embodiments, the carbohydrate base may be used from the carbohydrate source without purification. In such embodiments, the carbohydrate source is directly utilized for subsequent derivation steps.

In certain embodiments, gums suitable for use herein include any of those typically used in the appropriate application. For example, in food applications, useful gums include, but are not limited to alginates, guar gum, locust bean gum, xanthan gum, carrageenan, pectin, methyl cellulose, hydroxypropyl cellulose, and mixtures thereof. For cosmetics and pharmaceuticals, useful gums include, but are not limited to gum arabic, alginates, pectinate, carrageenan, carboxymethyl cellulose, hydroxypropyl cellulose, methylpropyl cellulose, and cellulose xanthate.

For use in sunscreen, useful gums include, but are not limited to, xanthan gum, guar gum, and alginate gums.

In certain embodiments, cellulose suitable for use herein includes any of those typically used in the appropriate application. For example, useful cellulose may include, but is not limited to cellulose, carboxymethyl cellulose, carboxymethyl hydroxyethylcellulose, hydroxypropyl methylcellulose, methyl hydroxyethylcellulose, microcrystalline cellulose, sodium cellulose sulphate, and hydroxyethylcellulose.

In certain embodiments, the carbohydrate base is treated using a treating agent. Such treatment yields a “carbohydrate derivative” which comprises one or more anionic moieties. In certain embodiments, the anionic moieties exist in a larger amount than which naturally occurs on the carbohydrate base. In other embodiments, the anionic moieties do not occur naturally on the carbohydrate base. In further embodiments, the anionic moieties are a combination of one or more different anionic moieties. In certain non-limiting embodiments, the one or more anionic moieties is a carboxylate, sulfonate, phosphate, or mixture thereof In some embodiments, the anionic moiety is carboxylate.

In still other embodiments, the treating agent is used at levels of about 0.1 to 17% based on the weight of carbohydrate. In further embodiments, the treating agent is used at levels of about 0.1 to 15%, 0.1 to 13%, 0.1 to 10%, 0.1 to 5%, 0.1 to 3%, or 0.1 to 1%. In yet further embodiments, the treating agent is used at levels of about 0.2 to 5%, or 0.2 to 4%, or 0.2 to 3%, or 0.2 to 2%, or 0.2 to 1%. In still yet other embodiments, the treating agent is used at levels of about 0.3 to 5%, or 0.3 to 4%, or 0.3 to 3%, or 0.3 to 2%, or 0.3 to 1%. In even still further embodiments, the treating agent is used at levels of about 0.3 to 1.8%, or 0.3 to 1.6%, or 0.3 to 1.4%, or 0.3 to 1.2%. In yet still other embodiments, the treating agent is used at levels of about 0.4 to 17%, 0.4 to 15%, 0.4 to 13%, 0.4 to 10%, 0.4 to 5%, or 0.4 to 4%, or 0.4 to 3%, or 0.4 to 2%, or 0.4 to 1%. In even still further embodiments, the treating agent is used at levels of about 0.4 to 1.8%, or 0.4 to 1.6%, or 0.4 to 1.4%, or 0.4 to 1.2% based on the weight of carbohydrate.

In determining the amount or level of treating agent required for treatment, consider, for example, a carbohydrate being treated with bleach at levels of about 0.8% active chlorine on weight of starch. The first step is to determine the amount of active chlorine of bleach. The carbohydrate is then treated at 0.8% active chlorine based on weight of the starch. Therefore, 0.8% active chlorine on 100g starch sample is 0.8g of active chlorine. If using sodium hypochlorite that has 10% active chlorine, then 8g of that 10% active chlorine sodium hypochlorite is needed to deliver 0.8g of active chlorine.

In certain embodiments, the treating agent is sodium hypochlorite. Sodium hypochlorite is useful for producing carbohydrate derivatives comprising carboxylate anionic moieties. In other embodiments, sodium hypochlorite is used at levels which yields active chlorine of about 0.1 to 5% based on weight of carbohydrate. In still other embodiments, sodium hypochlorite is used to treat at levels of active chlorine of 0.1 to 4%, or 0.1 to 3%, or 0.1 to 2%, or 0.1 to 1% based on weight of carbohydrate. In yet still other embodiments, the sodium hypochlorite is used to treat at levels of active chlorine of about 0.2 to 5%, or 0.2 to 4%, or 0.2 to 3%, or 0.2 to 2%, or 0.2 to 1%. In further embodiments, sodium hypochlorite is used to treat at levels of active chlorine of about 0.3 to 5%, or 0.3 to 4%, or 0.3 to 3%, or 0.3 to 2%, or 0.3 to 1%. In even further embodiments, sodium hypochlorite is used to treat at levels of active chlorine of about 0.3 to 1.8%, or 0.3 to 1.6%, or 0.3 to 1.4%, or 0.3 to 1.2%. In even yet still further embodiments, sodium hypochlorite is used to treat at levels of active chlorine of about 0.4 to 5%, or 0.4 to 4%, or 0.4 to 3%, or 0.4 to 2%, or 0.4 to 1%. In yet other embodiments, sodium hypochlorite is used to treat at levels of active chlorine of about 0.4 to 1.8%, or 0.4 to 1.6%, or 0.4 to 1.4%, or 0.4 to 1.2%. In still other embodiments, sodium hypochlorite is used to achieve about 0.1-1.0% of exposed carboxylate. In other embodiments, sodium hypochlorite is used to achieve about 0.1-0.8% of exposed carboxylate. In even further embodiments, sodium hypochlorite is used to achieve 0.1-0.5% of exposed carboxylate. In other embodiments, sodium hypochlorite is used to achieve about 0.1-0.3% of exposed carboxylate. In still other embodiments, sodium hypochlorite is used to achieve about 0.1-0.25% of exposed carboxylate. In yet even further embodiments, sodium hypochlorite is used to achieve about 0.1-0.2% of exposed carboxylate.

In certain embodiments, the treating agent is 1-chloro-2-sulfopropionic acid (CSPA). CSPA is useful for producing carbohydrate derivatives comprising sulfonate anionic moieties. In certain embodiments, CSPA is used to achieve about 0.1-1.0% of bound sulfonate. In certain embodiments, CSPA is used to achieve about 0.15-0.8% of bound sulfonate. In certain embodiments, CSPA is used to achieve 0.15-0.5% of bound sulfonate. In certain embodiments, CSPA is used to achieve about 0.15-0.3% of bound sulfonate. In certain embodiments, CSPA is used to achieve about 0.15-0.25% of bound sulfonate. In certain embodiments, CSPA is used to achieve about 0.15-0.2% of bound sulfonate.

In certain embodiments, the treating agent is sodium tripolyphosphate (STPP). STPP is useful for producing carbohydrate derivatives comprising phosphate anionic moieties. In certain embodiments, STPP is used to achieve about 0.1-0.5% of bound phosphate. In certain embodiments, STPP is used to achieve about 0.15-0.4% of bound phosphate. In certain embodiments, STPP is used to achieve about 0.15-0.35% of bound phosphate. In certain embodiments, STPP is used to achieve about 0.15-0.3% of bound phosphate. In certain embodiments, STPP is used to achieve about 0.15-0.25% of bound phosphate. In certain embodiments, STPP is used to achieve about 0.15-0.2% of bound phosphate.

In certain embodiments, the carbohydrate derivative is further modified with octenylsuccinate anhydride (OSA). In certain embodiments, the modification is obtained by mixing carbohydrate derivative with OSA yielding a carbohydrate derivative esterified with octenyl succinic acid. In certain embodiments, OSA is used at a final concentration of about 1 to about 7%, or about 1 to about 6%, or about 1 to about 5%, or about 1 to about 4% based on weight of carbohydrate. In certain embodiments, OSA is used at a final concentration of about 2 to about 7%, or about 2 to about 6%, or about 2 to about 5%, or about 2 to about 4% based on weight of carbohydrate. In certain embodiments, OSA is used at a final concentration of about 3 to about 7%, or about 3 to about 6%, or about 3 to about 5%, or about 3 to about 4% based on weight of carbohydrate.

In certain embodiments, the carbohydrate derivative modified with OSA is crosslinked with a polyvalent cation. In certain embodiments, the polyvalent cation is selected from the group consisting of metal or alkaline earth metal ions. In certain embodiments, the polyvalent cation is selected from the group consisting of calcium, zinc, iron, copper, and titanium. In certain embodiments, the polyvalent cation is divalent. The divalent cation may be selected from calcium, zinc, iron, copper, magnesium, and titanium. In certain embodiments, the divalent cation is calcium. In certain embodiments, the polyvalent cation is trivalent. The trivalent cation may be selected from aluminum, cobalt, and iron.

In certain embodiments, the octenylsuccinate modified carbohydrate derivative salts comprise OSA in amounts of about 0.1% to about 99%, or about 0.1% to about 90%, or about 0.1% to about 75%, or about 0.1% to about 50%, or about 0.1% to about 25%, or about 0.1% to about 10%, or about 0.1% to about 5%, or about 0.1% to about 3%, or about 1% to about 90%, or about 1% to about 75%, or about 1% to about 50%, or about 1% to about 25%, or about 1% to about 10%, or about 1% to about 6%, or about 1% to about 4%, or about 2% to about 3%.

Ordinary carbohydrates, such as starches, when commercially dry, do not flow freely but, rather, tend to agglomerate into clumps or cakes. In other words, the individual starch granules tend to stick to one another and agglomerate into larger masses, thus retarding flow and ease of movement. Alternatively, free flowing modified carbohydrate may exhibit an ease of flow that is comparable to a liquid. For example, placing a quantity of dry product of a free-flowing starch into a jar and shaking the jar may move the starch with a liquid-like motion, whereas an ordinary starch falls about in clumped masses. Further, when placing ordinary starch in a standard separatory funnel having a tube of about 6 mm inner diameter, the ordinary starch will not pour through the tube even when the funnel is shaken strongly. On the other hand, a major proportion of free-flowing starch product, when put under the same conditions, will pour from the funnel, even when the funnel is kept absolutely still. This notable free flowing property is of great importance for many applications.

In a composition, determination of water repellency may be made by examining the amount of the composition that settles to the bottom of an aqueous solution, such as water. To determine water repellency, the composition is placed in a container holding an aqueous solution, gently mixed into the aqueous solution, and allowed to settle over a period of time. The amount or volume of the material that settles to the bottom of the container is quantified. Settled material that occupies a smaller volume of space is considered water repellant, while settled material that occupies a larger volume of space is considered less water repellant or not water repellant.

One such test for water repellency is provided: briefly, 5.00 grams of sample was added to 75 mL of purified water in a 100 mL Goetz type centrifuge tube. The tube was gently inverted 10 times, without shaking, and allowed to sit at room temperature for 1 hour. The tube was then inverted ten additional times, allowed to settle for 15 minutes, and the amount of material which settled to the bottom was read and recorded as average amount settled after 1.25 hours. A value less than 2 mL is considered water repellent and a value greater than 5 mL is considered as not water repellant.

In certain embodiments, the octenylsuccinate modified carbohydrate derivative salt provides a volume of settled material of about 0 mL to about 5 mL, or about 0 mL to about 4 mL, or about 0 mL to about 3 mL, or about 0 mL to about 2.5 mL, or about 0 mL to about 2.0 mL, or about 0 mL to about 1.5 mL, or about 0 mL to about 1 mL, or about 0 mL to about 0.5 mL, about 0.1 mL to about 5 mL, or about 0.1 mL to about 4 mL, or about 0.1 mL to about 3 mL, or about 0.1 mL to about 2.5 mL, or about 0.1 mL to about 2.0 mL, or about 0.1 mL to about 1.5 mL, or about 0 mL to about 1 mL, or about 0 mL to about 0.5 mL.

Determination of free flow may also be made by examining the angle of repose of a powder. To determine the angle of repose, powder is first poured from an elevation onto a flat surface followed by determining the angle that the cone-like powder slope makes with respect to the horizontal surface. This angle is qualified as the angle of repose. A smaller angle of repose generally implies enhanced free flow characteristics.

In some embodiments, the octenylsuccinate modified carbohydrate derivative salt has an angle of repose of about 5 to about 85 degrees, or about 15 to about 75 degrees, or about 15 to about 60 degrees, or about 15 to about 45 degrees, or about 15 to about 30 degrees, or about 25 to about 75 degrees, or about 25 to about 60 degrees, or about 25 to about 45 degrees, or about 25 to about 30 degrees, or about 15 degrees, or about 20 degrees, or about 25 degrees, or about 30 degrees, or about 35 degrees, or about 40 degrees, or about 45 degrees.

Compositions described herein provide carbohydrate-based molecules that are free flowing, oil absorbing and repel water. Such compositions are useful in applications that require such properties, such as compositions for applications in skin care, hair care, cosmetics, food ingredients, dusting and lubrication agent, and dry wall components. In some aspects, such compositions are useful as an oil absorbent in powder formulations. In some aspects, such compositions are useful as a cosmetic. In some aspects, such compositions are useful as an oil or grease absorbent. In some aspects, such compositions are useful as an oil absorbent in dry shampoo. In some aspects, such compositions are useful as conditioners. In certain embodiments, such compositions are useful in a sunscreen composition. In some aspects, such compositions are useful in a food composition. In still other aspects, such compositions are useful in an industrial application.

In certain embodiments, the octenylsuccinate modified carbohydrate derivative salts can be used in different cosmetic compositions, such as in skin care and antiperspirants, where they provide free flow, oil absorption and water repellant properties. In certain embodiments, such cosmetic compositions include, but are not limited to, facial moisturizers, body lotions, facial treatments, foundations, eye shadows, highlighters, blushes, facial powders, face primers, lipsticks, and lip glosses, and lip balms.

Cosmetic skin care compositions may involve different media or systems and comprise a suitable cosmetic vehicle or base for the composition. This vehicle may be an emulsion, an aqueous system, a solvent system or a combination of aqueous and solvent systems as well as anhydrous and powdered systems.

In certain embodiments, emulsions are the preferred vehicle or base for cosmetic compositions of this technology and products of this type include skin care creams and lotions. These emulsions, which comprise water-based and oil-based phases, may be oil-in-water emulsions having oil as the dispersed phase and water as the continuous phase or they may be water-in-oil emulsions with water dispersed in oil, which is the continuous phase. The oil phase, which may comprise from about 10 to 90% by weight of the composition, is typically made up of cosmetically acceptable or conventional oily substances that are soluble in this phase, such as oils, waxes and emulsifiers. Compounds which can be included in the oil phase are typically mineral, animal and vegetable oils and fats, synthetic esters, fatty acids, aliphatic alcohols, higher fatty alcohols, alkyl amines, waxes, so called mineral fats and oils, such as paraffin oil, petrolatum, ceresin, silicone oils and silicone fats. The water phase may comprise from about 10 to 90% water by weight of the composition and include water soluble components such as alkalis, alkanolamines, polyhydric alcohols and preservatives. These emulsions may include one or more emulsifiers which usually are contained in the oil phase but in some instances, depending on the type, may be in the water phase. Emulsifiers which can be used may be ionic or nonionic, are well known and constitute a large group of conventional and commercially available products. They are often characterized by their hydrophilic-lipophilic balance (HLB). Oil-in-water (0/W) emulsifying agents typically have an HLB of more than 6.0 and produce emulsions in which the continuous phase is hydrophilic and such emulsions are generally dispersible in water. Emulsifiers of this type include PEG 300 distearate, sorbitan monolaurate and triethanolamine stearate. Water-in-oil (W/O) emulsifiers usually have an HLB of less than 6.0, preferably below 5, and produce emulsions in which the continuous phase is lipophilic. Such emulsifiers include, lanolin alcohols, ethylene glycol monostearate, sorbitan mono-oleate and PEG 200 dilaurate. Emulsifiers with HLB's of between 5 and 7 may function as either W/O or O/W emulsifiers depending on how they are used.

The amount of emulsifier used can vary depending on the system and typically will be an effective emulsifying amount. In certain embodiments, the amount of emulsifier can vary from about 0.1 to 20% by weight of the composition and preferably from about 0.2 to 10%.

Various other ingredients and additives may be included in one or both of the oil and water phases in the cosmetic skin care emulsions described above. This includes emollients, humectants, thickening agents, UV-light inhibitors, preservatives, pigments, dyes, colorants, alpha hydroxy acids, aesthetic enhancers such as perfumes and fragrances, film formers (water proofing agents), antiperspirants, deodorants, antiseptics, antifungal, antimicrobial and other medicaments and solvents. Effective amounts of one or more of these and other active and functional ingredients are generally used and this can total from about 0.1 to 25% by weight of the composition and more particularly from about 0.1 to 15%.

Other cosmetic compositions using the octenylsuccinate modified carbohydrate derivative salt involves aqueous or solvent systems wherein the added components are soluble or dispersible therein. The aqueous system may comprise octenylsuccinate modified carbohydrate derivative salt in addition to additives and active and functional ingredients, optionally a propellant and the balance water. Generally, an aqueous system will comprise from about 10 to 99.8% by weight water, preferably about 50 to 80%; from about 0.1 to 20% by weight of the octenylsuccinate modified carbohydrate derivative salt, preferably 0.2 to 10%; from about 0.1 to 25% by weight of additives and ingredients, preferably 0.1 to 15%; and from about 0 to 50% by weight of propellant, preferably 0 to 30%. Compositions of this type include the topical sprays and products containing fragrances and antimicrobial agents.

In certain embodiments, the cosmetic may be a topical spray. Topical sprays may include aerosol sprays or products containing a propellant. While any of the known propellants may be used in the compositions of this invention, preferred propellants included the non-halogenated hydrocarbons, particularly the lower boiling hydrocarbons such as C3-C6 straight and branched chain hydrocarbons, i.e., propane, butane, isobutane and mixtures thereof. Other preferred propellants include the ethers, such as dimethyl ether, hydrofluorocarbon and the compressed gases such as N2 and CO2.

Certain embodiments may make use of a solvent system as the vehicle or base involves other cosmetic compositions containing the selected octenylsuccinate modified carbohydrate derivative salt. The solvent system may comprise octenylsuccinate modified carbohydrate derivative salt in addition to additives and active and functional ingredients, optionally a propellant and the balance solvent. The solvent may be any of the known organic solvents which may solubilize or disperse components of the skin care composition and more particularly aliphatic alcohols, esters, ethers, ketones, amines and hydrocarbons including the aromatic, nitrated and chlorinated hydrocarbons. Particularly preferred organic solvents are the lower aliphatic alcohols such as the C1-3 alcohols and especially ethanol. Generally, the solvent system may comprise from about 25 to 99.8% by weight of solvent, preferably about 50 to 80%; from about 0.1 to 20% by weight of the octenylsuccinate modified carbohydrate derivative salt, or preferably 0.2 to 10%; from about 0.1 to 25% by weight of additives, or preferably from about 0.1 to 15%; and from about 0 to 75% by weight of propellant, preferably about 0 to 35%.

The additives and other ingredients which may be included in either the aqueous or solvent based systems are the same as those described above for the emulsion and oil based systems. The propellants which may be included in the solvent system are the same as those described above for the aqueous systems. Additionally, a mixture of the aqueous and solvent systems may be used wherein water and solvent, especially alcohols are combined along with the components, i.e., octenylsuccinate modified carbohydrate derivative salt, additives and propellant. Such a composition may comprise about 25 to 99.8% by weight of the composition of a combination of water and solvent, preferably about 50 to 80% along with the components as described above.

Anhydrous and powdered systems may also be used incorporating the octenylsuccinate modified carbohydrate derivative salt of this invention. The anhydrous system may comprise as a base, materials such as mineral oil, wax, esters, volatile solvents, etc., and will also include the selected octenylsuccinate modified carbohydrate derivative salt, additives and active and functional ingredients. Powdered systems may comprise, as a base powder material, corn starch and mica, fillers such as clay as well as other additives and active and functional ingredients. Generally, the anhydrous system may comprise from about 10 to 99.8%, preferably 10 to 90% by weight of base material; from about 0.1 to 65%, preferably 0.5 to 40% by weight of octenylsuccinate modified carbohydrate derivative salt; and from about 0.1 to 25%, preferably 0.1 to 15% by weight of additives. The powdered system may comprise from 0 to 99.8%, preferably 1 to 90% by weight of the powder material; from about 0.1 to 99%, preferably 0.5 to 90% by weight of the octenylsuccinate modified carbohydrate derivative salt; and from about 0.1 to 25%, preferably about 0.1 to 15% by weight of additives.

In certain embodiments, cosmetic compositions may comprise octenylsuccinate modified carbohydrate derivative salt from about 0.1 to 99% and preferably from about 0.2 to 90% by weight, based on the weight of the composition. As noted earlier when using emulsion, aqueous and solvent systems the amount of octenylsuccinate modified carbohydrate derivative salts may comprise from about 0.1 to 20% and preferably from about 0.2 to 10% by weight, based on the weight of the composition. When using anhydrous systems, the amount of octenylsuccinate modified carbohydrate derivative salt may comprise from about 0.1 to 65% and preferably 0.5 to 40% by weight. When using powdered systems, the amount of octenylsuccinate modified carbohydrate derivative salt may comprise from about 0.1 to 99% and preferably from about 0.5 to 90% by weight.

In certain embodiments, cosmetic compositions containing octenylsuccinate modified carbohydrate derivative salt in accordance with this invention may further comprise an effective aesthetic enhancing additive. This can vary widely depending on the application.

Preparation of cosmetic emulsion compositions typically involves adding oil soluble components in one vessel and heating to, e.g., about 75° to 8 about 0° C., and combining the water soluble components in another vessel and heating to, e.g., about 75° to about 80° C. Depending on whether 01W or W/O emulsions are being prepared, the warmed inner phase may then slowly be added to the outer phase with agitation.

Some embodiments are directed to food compositions made with compositions comprising octenylsuccinate modified carbohydrate derivative salt in addition to also having at least one additional edible ingredient. Certain exemplary edible ingredients may include, but are not limited to, proteins or protein hydrolysates, vegetable oils, other carbohydrates, pharmaceutically acceptable carriers, and other food additives such as those described below. In certain embodiments, illustrative edible ingredients may include, but are not limited to, egg yolk, sweeteners (e.g. sucrose, rebaudioside, allulose, corn syrup, glucose), salts, seasonings commonly used in edible compositions, dairy ingredients whether liquid (milk, cream), solid (cheese, butter, non-fat milk solids, whey protein, casein) oils commonly used in edible emulsions including flavoring oils, and cooking oils (olive oil, corn oil, coconut oil, safflower oil, canola oil), flavoring extracts, flavoring syrups, water, vinegar or other acids (liquid or powdered), alcohol, fiber and fiber derived products, starches, gums, other edible hydrocolloids.

In some embodiments, the food compositions described herein may be in solid or liquid form. In some embodiments, the food compositions described herein may take the form of a dry powder and include at least one salt or acid.

The type of food composition is not limited, but in various embodiments, the food composition comprising octenylsuccinate modified carbohydrate derivative salt is one or more of the following: sauces, dressings, ice creams and frozen desserts, beverages, yogurts, bakery fillings and glazes, puddings, custards, or other cream or cream like desserts and dessert toppings, and thickened fruit preparations, cheese or cheese-like or analog cheese spreads and other edible spreads.

In certain embodiments, the food compositions described herein may include a variety of additional synthetic and natural additives and components. The particular additives and components used will depend on the nature of the desired end product. However, by way of illustration, examples of the types of ingredients that may be included in the various food compositions described herein are provided below.

In some embodiments, octenylsuccinate modified carbohydrate derivative salts described herein can be used alone or in combination with other saccharides, oligosaccharides, or carbohydrates. Examples of further oligosaccharides include short-chain fructooligosaccharides, isomaltooligosaccharides, maltodextrin, and inulin. The food compositions of the present technology may further comprise another carbohydrate component. Examples of carbohydrate components include, but are not limited to, sucrose, high-fructose corn syrup, dextrose, hydrolyzed starch, polymerized glucose, maltose, glucose, lactose, fructose or combinations thereof.

In certain embodiments, the food compositions described herein may include a taste-improving compound. A taste-improving compound produces a food composition having a more sugar-like taste or a sugar-like temporal profile than would be experienced if the taste improving composition were not included in the food composition. The taste-improving compositions include, but are not limited to, polyols, amino acids and their corresponding salts, polyamino acids and their corresponding salts, sugar acids and their corresponding salts, organic acids, inorganic acids, organic salts, inorganic salts, bitter compounds, flavorants, astringent compounds, polymers, proteins or protein hydrolysates, surfactants, emulsifiers, flavonoids, alcohols, synthetic sweeteners, and combinations thereof

In certain embodiments, the food composition described herein may comprise natural high-intensity sweeteners. Examples of natural high-intensity sweeteners include, but are not limited to, rebaudioside A, rebaudioside B, rebaudioside C, rebaudioside D, rebaudioside E, rebaudioside F, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, and combinations thereof

In certain embodiments, the food compositions described herein may further comprise a synthetic high-intensity sweetener. Examples of synthetic high- intensity sweeteners include, but are not limited to, sucralose, acesulfame potassium and other salts, aspartame, alitame, saccharin, neohesperidin dihydrochalcone, cyclamate, neotame, N-[1\1-[3-(3-hydroxy-4-methoxyphenyl)propyl]-L-a-aspartylR-phenylalanine-1-methylester, N4N-[3-(3-hydroxy-4-methoxyphenyl)-3-methylbutyl]-L-a-asp artylR-phenylalanine- 1 -methyl ester, N-[1\143-(3-methoxy-4-hydroxyphenyl)propylR-a-aspartylR-phenylal-anine-1-methylester, salts thereof, and combinations thereof. However, some embodiments of the food compositions described herein will be free of synthetic sweeteners.

In certain embodiments, the food compositions described herein may further comprise antioxidants. Types of antioxidants include ascorbic, isoascorbic, erythorbic, and citric acid types. Other examples include sodium, calcium, and potassium ascorbate; soybean lecithin; esters of citric acid and fatty acids with glycerol; esters of citric and mono- and di- glycerides; enzymes such as glucose-oxidase; ascorbyl palmitate, ascorbyl stearate; a concentrated mix of tocopherols or tocopherols and alpha-tocopherol; propyl galate; tert-butyl hydroquinone (TBHQ); butyl hydroxyanisol (BHA); butyl hydroxytoluene (BHT); isopropyl citrate (mix); and isopropyl citrate (mono).

In certain embodiments, the food compositions described herein may further comprise a conservant. Examples of conservants include, but are not limited to, propionic and acetic acid;

sodium, calcium, and/or potassium propionate; sodium erythorbate; isoascorbate; and calcium acetate.

In certain embodiments, the food compositions described herein may further comprise an emulsifier/stabilizer. Examples of emulsifiers and stabilizers include, but are not limited to, propylene glycol alginate, polyethylene stearate, sorbitan derivatives (polyoxyethylene stearate, polyoxyethylene monooleate, polyoxyethylene monolaurate, polyoxyethylene monopalmitate, polyoxyethylene monostearate, polyoxyethylene tristearate, stearate, monooleate, tristearate, monopalmitate), sodium stearoyl-2-lactylate, calcium stearoyl-2-lactylate, fatty acid esters with propylene glycol, tartaric diacetyl acid esters and fatty acids with glycerol, tartaric diacetyl acid esters and mono and diglycerides, lecithin, sodium caseinate, citrate (sodium, monosodium, disodium and trisodium), gums (xanthan, guar, adragante, arabic, konjac), mono and diglycerides, sorbitol, cellulose derivates (methyl, methyl ethyl, hydroxypropyl, microcrystaline), sodium carboxymethyl cellulose, and salts of fatty acids (calcium, sodium, potassium, ammonium).

In certain embodiments, the food compositions described herein may further comprise a polyol. Examples of polyols include, but are not limited to, erythritol, xylitol, sorbitol, maltitol, lactitol, mannitol, isomalt, polydextrose, and hydrogenated starch hydrolysates or combinations thereof

In certain embodiments, the food compositions described herein may further comprise a flavor enhancer. Examples of flavor enhancers include, but are not limited to, glutamic acid and its salts, guanilic acid and its salts, inosinic acid and its salts, or combinations thereof.

In certain embodiments, the food compositions described herein may further comprise bulking agents. The term “bulking agents,” as used herein, may be any of those typically used in the art and include polydextrose, cellulose and its derivatives, maltodextrin, corn syrup solids, sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, pectin, alginate, gum arabic, xanthan, guar, gellan, carrageenan, gelatin, starch, modified starch, and the like, or combinations thereof

Still other embodiments are directed to sunscreen products made with compositions comprising octenylsuccinate modified carbohydrate derivative salt, in addition to also having at least one additional ultraviolet absorbing agent, ultraviolet scattering agent, or both. Such ultraviolet absorbing or ultraviolet scattering agents are used to achieve desirable levels and effects of protecting the skin from ultraviolet light. Ultraviolet-absorbing agents are usually organic compounds, such as ethylhexyl methoxycinnamate and octocrylene. On the other hand, ultraviolet-scattering agents include inorganic, powder components, such as titanium dioxide and zinc oxide. In certain embodiments, sunscreen products are made with compositions comprising octenylsuccinate modified carbohydrate derivative salt combined with specific organic ultraviolet absorbing agents to achieve different sun protection factor (SPF) numbers. Examples of SPF numbers achieved in sunscreens and other formulations providing sunscreen protection include, but are not limited to, 5, 10, 15, 20, 30, 50, and 100.

In certain embodiments, the sunscreen may be formulated to be sprayable. In certain embodiments, the sunscreen may be formulated to be a lotion.

Any sunscreen active known in the art, or combination thereof, may be used in the compositions described herein. The term “sunscreen active” is intended to include any ultraviolet ray-blocking compounds exhibiting absorption or blockage within the wavelength region between about 290 and 420 nm, or infrared radiation. Sunscreen actives are suitably classified into five groups based upon their chemical structure: amino benzoates; salicylates; cinnamates; benzophenones; and miscellaneous chemicals including menthyl anthranilate and digalloyl trioleate. Inorganic sunscreens may also be used including titanium dioxide, zinc oxide, iron oxide, and polymer particles such as those of polyethylene and polyamides. Examples of suitable aminobenzoic acids include aminobenzoic acid, its salts, and its derivatives, such as ethyl, isobutyl, and glyceryl esters, p-dimethylaminobenzoic acid, and 4-aminobenzoic acid derivatives, including 4-(dimethylamino)-benzoic acid-2-ethylhexyl ester, 4-(dimethylamino)-benzoic acid-2-octyl ester and 4-(dimethylamino)-benzoic acid amyl ester, and the like. Examples of suitable cinnamates include cinnamic acid derivatives such as methyl and benzyl esters, a-phenyl cinnamonitrile, butyl cinnamoyl pyruvate, and the like.

In exemplary embodiments, the sunscreen actives are FDA approved or approved for use in the European Union. Examples of suitable FDA approved sunscreen actives are described in the Final Over-the-Counter Drug Products Monograph on Sunscreens, herein incorporated by reference. It should be understood that the specific sunscreen actives and amounts thereof that are approved for use in the United States or European Union are subject to periodic change. As such, the specific examples and amounts set forth herein are not intended to be limiting.

Another embodiment is directed to body powder compositions comprising OSA modified carbohydrate derivative salts of the invention. The OSA modified carbohydrate derivative salts of the present invention may act as carriers within the body powder. In certain embodiments, the carrier typically comprises from about 25% to about 99%, preferably from about 30% to about 80%, more preferably from about 35% to about 75%, and most preferably from about 40% to about 70%, by weight of the body powder. Preferably, where the powder carrier comprises talc, talc comprises less than about 50% by weight of the body powder. Where a body powder is intended for use in the panty area, preferably the powder comprises less than 5%, preferably less than 3%, most preferably, about 0% of talc, by weight of the body powder.

Such body powders may comprise moisture absorbers to aid in reducing excess moisture, particularly on occluded skin. As used herein, the phrase “moisture absorbers” refers to silicas (or silicon dioxide), silicates or carbonates. The silicates and carbonates are those formed by reaction of a carbonate or silicate with the alkali (IA) metals, alkaline earth (IIA) metals, or transition metals. Certain preferred are moisture absorbers in the form of microspheres and/or ellipsoids.

In some embodiments, the moisture absorbers described herein are included in the body powder such that the final body powder is capable of absorbing from about 0.8 grams of excess moisture per gram of body powder (0.8 g/g), to about 6.0 grams of excess moisture per gram of body powder (6.0 g g); more preferred from about 1.0 g/g to about 4.0 g/g; and most preferred from about 1.5 g/g to about 2.5 g/g.

Moisture absorbers useful in some embodiments include, for example, calcium silicate, amorphous silicas, calcium carbonate, magnesium carbonate, or zinc carbonate, and mixtures thereof. Some specific examples of the silicates and carbonates useful herein are more fully explained in Van Nostrand Reinhold's Encyclopedia of Chemistry. 4th Ed. pp. 155, 169, 556, and 849, (1984), which is incorporated herein by reference. In some embodiments, synthetic versions of the moisture absorbers are used, particularly in regard to silicas and silicates due to safety risks related to crystalline silica. Synthetic versions are formed by controlled chemical reactions in a manufacturing process rather than using a natural, mined version of these compounds which is then further refined. Synthetic carbonates useful herein can be obtained from various suppliers such as Mallinckrodt or Whittaker, Clark, and Daniels. Examples of synthetic calcium silicates useful in the present invention are Hubersorb® 250 or Hubersorbg 600 available from J.M. Huber. In some embodiments, the moisture absorbers comprise from about 2% to about 60%; from about 6% to about 60%; from about 16% to about 55%; from about 20% to about 50%; from about 25% to about 45%; and from about 35% to about 40% by weight of the body powder composition. Absorbent powders comprising mainly silicas for moisture control are preferred over those powders comprising mainly silicates and/or carbonates for moisture control. In some embodiments, the powders comprise silicas in the form of microspheres and/or ellipsoids, which have been found to contribute good skin feel characteristics in addition to efficient moisture absorption. Silica ellipsoids useful in the present invention are available from DuPont as ZELEC® Sil. Silica microspheres are available from KOBO as MSS-500, MSS 500/3, MSS-500/H, MSS-500/3H, MSS-500/N, and MSS-500/3N; from Presperse as Spheron L-1500, Spheron P-1000, Spheron P-1500; and from US Cosmetics as Silica Beads SB-300 and SB-700. Additionally, in embodiments where increased flowability of the powder is desired, at least some of the silica is fumed silica, which is available from Cabot Corporation (Cab-O- Sil®) and from Degussa (Aerosil®).

In certain embodiments, the body powder comprises additional components. Such components may include carbon odor-controlling agents, such as ones described in U.S. Pat. No. 5,429,628. Such carbon odor controlling agents may be used in the present invention at a level of from about 0.1% to about 25%, by weight of the body powder composition. Sodium bicarbonate, which is known in the art for its use as an odor absorber, may also be used. When included in the present invention, sodium bicarbonate may be present from about 0.1% to about 50%, by weight of the body powder composition. Antimicrobial agents may be used. Such agents may be selected from antibacterial agents, antifungal agents, and mixtures thereof. In some embodiments, the antimicrobial agents are selected from zinc phenolsulfonate, zinc oxide, triclosan, ^{Zelec}® AM by DuPont, zinc ricinoleate, zinc undecylenate, and mixtures thereof. In some embodiments, the antimicrobial agents are used at a level of from about 0.01% to about 25% or from about 0.1% to about 10%, by weight of the body powder composition.

In further embodiments, the body powder may comprise skin aids such as, for example, skin protectants, emollients, moisturizers, and antioxidants. Skin protectants useful in the embodiments described herein are set forth in the Cosmetic Bench Reference, 1994 Edition, page 53; and the Monograph on Skin Protectant Drug Products for Over-the-Counter Human Use, 21 CFR 347. In some embodiments, the skin protectant is selected from com starch, kaolin, mineral oil, sodium bicarbonate, dimethicone, zinc oxide, colloidal oatmeal, and mixtures thereof. When present, the skin protectants comprise from about 0.1% to about 80%, from about 0.1% to about 30%, or from about 0.1% to about 10%, by weight of the body powder composition.

In further embodiments, the body powder may further comprise one or more of emollients, moisturizers, antioxidants, binders, antipruritics, colors, fragrances, and preservatives.

Another embodiment is directed to antiperspirant compositions comprising OSA modified carbohydrate derivative salts. Such antiperspirants may include ingredients such as ones known in the art, for example, as found in the Cosmetic Bench Reference, 1994 Edition, page 13, which is incorporated herein by reference. For example, the antiperspirant composition may comprise non-amino functionalized silicone oils as the oil continuous phase. The silicone oils may be volatile or non-volatile. In some embodiments, the volatile oils are linear siloxanes containing from 3 to 9 silicon atoms, and cyclic siloxanes having from 4 to 6 silicon atoms such as cyclopentasiloxane. Examples of commercially available volatile silicone oils include oils having grade designations 344, 345, 244, 245 and 246 from Dow Corning Corporation. Certain suitable non-volatile silicone oils are polyalkylsiloxanes, in particular polydimethylsiloxanes which have the CTFA designation dimethicone that are commercially available under the name Dow Corning 556 and Dow Corning 200 series. In certain embodiments, the antiperspirant composition comprises the non-amino functionalized silicone oil in an amount ranging from 0.05 to 50%, from 1 to 40%, or from 5 to 30% by total weight of the antiperspirant composition. The antiperspirant composition may also comprise natural oils that comprise a glyceride of an unsaturated carboxylic acid containing 1, 2, or 3 olefinic bonds. In some embodiments, the unsaturated carboxylic acid containing 1, 2, or 3 olefinic bonds that is part of the glyceride contains from 14 to 22 carbon atoms, 16-20 carbon atoms, or 18 carbon atoms. In other embodiments, the amount of natural oil ranges from about 0.1 to 20%, 0.5 to 15%, or 1 to 10% by total weight of the antiperspirant composition.

Humectants may also be included in the antiperspirant composition. In some embodiments, the humectant is glycerine; sorbitol; propylene glycol; dipropylene glycol; diglycerol; triacetin; mineral oil; polyethylene glycol (preferably, PEG-400); alkane diols like butane diol and hexanediol; ethanol; pentylene glycol; or a mixture thereof. In other embodiments, the humectants are employed in an amount from 0.01 to 20%, 0.1 to 10%, or 0.5 to 5% by total weight of the antiperspirant composition.

The antiperspirant composition may further comprise fragrance. Exemplary fragrances include perfumes as described in European Patent No. 545,556. In some embodiments, the amount of fragrance contained in the antiperspirant ranges from about 0.01 to 4%, 0.1 to 3%, 0.25 to 2% by total weight of the antiperspirant composition.

The viscosity of the antiperspirant composition is typically at least about 8000 cP (1 cP=1 mPas) at 25° C., at least about 8500 cP, or from about 8500 cP to about 15000 cP. The viscosity can be measured by DV-I Viscometer (Brookfield Ltd).

Still another embodiment is directed to shampoo compositions comprising OSA modified carbohydrate derivative salts. In some embodiments, the shampoo composition is a dry shampoo composition. Such shampoo may include OSA modified carbohydrate derivative salt material, a hydrophobic emollient, particularly a long chain alkane, and optionally a propellant. The dry shampoo composition can further comprise a fragrance. In some embodiments, the dry shampoo is substantially free of silicones. In various embodiments, the dry shampoo is substantially free of petroleum based cationic surfactants. In certain embodiments, the dry shampoo is substantially free of distearyldimonium chloride.

In some embodiments, the weight percentage of OSA modified carbohydrate derivative salt material in the dry shampoo composition, based on the total weight of the dry shampoo composition, can be about 0.1% to about 15%, about 1% to about 12%, about 2% to about 10%, or about 4% to about 8%. In certain embodiments, the weight percentage of OSA modified carbohydrate derivative salt material in the dry shampoo composition, based on the total weight of the dry shampoo composition, can be at least about 0.1%, at least about 1%, or at least about 4%, preferably with an upper range limit of about 50% by weight.

In various embodiments, the dry shampoo composition can comprise an emollient useful for moisturizing the hair and thus acting as a conditioning agent or styling agent. The emollient particularly is a hydrophobic emollient. For example, the emollient of the dry shampoo composition can comprise a long chain alkane. As used herein, a long chain alkane is at least a Cio alkane or at least a C12 alkane, preferably up to a C40 alkane. For example, in certain embodiments the dry shampoo composition can comprise an emollient in the form of a

C13-C15 alkane. In some embodiments, the emollient comprises a squalane and/or one or more derivatives thereof, particularly hemisqualane.

In some embodiments, the dry shampoo composition can comprise one or more additional conditioning agents known in the art, in addition to one or more hydrophobic emollients. Additional conditioning agents known in the art include, but are not limited to, silicones (e.g., phenyl trimethicones, dimethicones, cyclomethicones, dimethicone copolyols, amino silicones, etc.), petroleum based cationic surfactants, distearyldimonium chloride, guar compounds including cationic polymers and guar gum.

Some embodiments are directed to methods of using the compositions described herein. In other embodiments, methods of using octenylsuccinate modified carbohydrate derivative salt compositions comprises inclusion of octenylsuccinate modified carbohydrate derivative salt within a food and consumption of the food. In certain embodiments, methods of use of octenylsuccinate modified carbohydrate derivative salt compositions comprises inclusion of octenylsuccinate modified carbohydrate derivative salt within a cosmetic and applying the cosmetic. In another embodiment, methods of use of octenylsuccinate modified carbohydrate derivative salt compositions comprises inclusion of octenylsuccinate modified carbohydrate derivative salt within a shampoo and use of the shampoo. In another embodiment, methods of use of octenylsuccinate modified carbohydrate derivative salt compositions comprises inclusion of octenylsuccinate modified carbohydrate derivative salt within an antiperspirant and use of the antiperspirant. In another embodiment, methods of use of octenylsuccinate modified carbohydrate derivative salt compositions comprises inclusion of octenylsuccinate modified carbohydrate derivative salt within an ointment and applying the ointment. In a further embodiment, methods of use of octenylsuccinate modified carbohydrate derivative salt compositions comprises inclusion of octenylsuccinate modified carbohydrate derivative salt within a sunscreen and applying the sunscreen. In certain embodiments, composition use is as a component of a food, cosmetic, dry shampoo, anti-perspirant, deodorant, ointment or sunscreen.

In certain embodiments, the invention includes methods of preparing a composition comprising an octenylsuccinate modified carbohydrate derivative salt, said method comprising treating a carbohydrate base to produce a carbohydrate derivative; esterifying the carbohydrate derivative with octenyl succinic anhydride to produce a carbohydrate derivative esterified with octenyl succinic acid; and mixing the carbohydrate derivative esterified with octenylsuccinate anhydride with a polyvalent cation. In certain embodiments, prior to mixing with the polyvalent cation, the composition is at pH of about 8. In certain embodiments, after mixing with the polyvalent cation, the composition is at pH of about 7. In certain embodiments, the carbohydrate base is oxidized by treatment with sodium hypochlorite. In other embodiments, the carbohydrate base is treated with sodium hypochlorite at levels of about 0.4 to 1.2% active chlorine based on weight of carbohydrate. In some embodiments, the alkyl or alkenyl succinate anhydride with which the derivatized starch is mixed is OSA and the mixing is performed using about 1 to 5% OSA based on weight of carbohydrate. In certain embodiments, mixing the derivatized starch with OSA is performed using about 3 to 5% OSA. In certain embodiments, mixing calcium with the starch derivative and octenylsuccinic anhydride composition uses calcium at about 4-20 times the stoichiometric equivalent to the anionic functional groups present on the carbohydrate.

Subject matter contemplated by the present disclosure is set out in the following numbered embodiments:

1. A modified carbohydrate, wherein the modified carbohydrate is produced from a carbohydrate base being:
a) derivatized with one or more anionic moieties;
b) esterified with octenylsuccinic acid; and
c) complexed with a polyvalent cation.

2. The modified carbohydrate of claim 1, wherein the one or more anionic moieties is selected from the group consisting of carboxylate, sulfonate, phosphate, and mixtures thereof.

3. The modified carbohydrate of any preceding claim, wherein the carbohydrate base is selected from starch, cellulose and gum; and optionally, wherein the carbohydrate base is starch sourced from the group consisting of corn, high amylose corn, waxy corn, potato, pea, rice, waxy rice, sago, tapioca, waxy tapioca, and mixtures thereof.

4. The modified carbohydrate of any preceding claim, wherein the polyvalent cation is selected from the group consisting of metal or alkaline earth metal ions; and optionally, wherein the polyvalent cation is selected from the group consisting of calcium, zinc, copper, iron, and titanium.

5. The modified carbohydrate of any preceding claim, wherein the angle of repose of the modified carbohydrate is between about 20 to about 35.

6. The modified carbohydrate of any preceding claim, wherein the carbohydrate base is starch, the functional anionic moiety is carboxylate and the polyvalent cation is calcium.

7. The modified carbohydrate of any preceding claim, wherein the modified carbohydrate has a property selected from the following: free flowing, oil absorbing and water repellant, and mixtures thereof.

8. A personal care, food, or industrial composition comprising the modified carbohydrate of any preceding claim;

optionally, wherein the composition is a cosmetic, sunscreen, lotion, dry shampoo, anti-perspirant, deodorant, body powder, ointment, conditioner.

9. A method of using the composition of claim 9 as an ingredient in a food, cosmetic, dry shampoo, anti-perspirant, deodorant, ointment, sunscreen or industrial application.

10. Use of the composition of claim 9 in a food, cosmetic, dry shampoo, anti-perspirant, deodorant, ointment or sunscreen application.

11. A method of preparing a modified carbohydrate comprising:
a) treating a carbohydrate base to produce a carbohydrate derivative;
b) esterifying the carbohydrate derivative with octenylsuccinic anhydride to produce a carbohydrate derivative esterified with octenyl succinic acid; and
c) mixing the carbohydrate derivative esterified with octenyl succinic acid with a polyvalent cation.

12. The method of claim 11, wherein the polyvalent cation comprises calcium;

optionally, wherein the polyvalent cation is selected from calcium acetate or calcium chloride; and

optionally, wherein prior to mixing with the polyvalent cation, the esterified carbohydrate derivative is at a pH of about 8.

13. The method of one of claims 11-12, wherein the treating step uses sodium hypochlorite at a level of about 0.1 to about 17%, about 0.1 to about 15%, about 0.1 to about 13%, about 0.1 to about 10%, about 0.1 to about 5%, about 0.1 to about 3%, about 0.1 to about 1%, or about 0.4 to about 1.2% active chlorine based on the weight of the carbohydrate base;

optionally, wherein the treating step uses sodium hypochlorite to treat at levels of about 0.4 to about 1.2% active chlorine based on the weight of the carbohydrate base.

14. The method of one of claims 11-13, wherein the esterifying step uses about 1% to about 5% of octenylsuccinic anhydride based on weight of carbohydrate.

15. The method of one of claims 11-14, wherein the mixing step uses calcium at 4-20 times the stoichiometric equivalent to the anionic functional groups present on the carbohydrate.

### EXAMPLES

The following examples further illustrate embodiments of this invention. In the examples, unless otherwise indicated, all parts and percentages are given by weight of the composition and all temperatures are in degrees Celsius.

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

Preparation of Carbohydrate Derivatives

Carbohydrate derivatives having carboxylate anionic moieties was prepared using potato, tapioca, waxy corn, or dent corn as starch base materials. To prepare this derivatized carbohydrate, corn starch was added to a water solution at 25° C. and pH 8.5. The combination was mixed while maintaining pH with use of 3% (w/w) NaOH. Next, sodium hypochlorite was added to achieve a final concentration of 0.8% active chlorine, based on anhydrous starch amount. The reaction was maintained for 2 hours with constant agitation, providing a carboxylated carbohydrate derivative.

Carbohydrate derivatives having phosphate anionic moieties was prepared using dent corn as starch base material. To prepare this derivatized carbohydrate, sodium tripolyphosphate (STPP) was dissolved into water to achieve a final STPP amount of 14% weight to starch weight and total solution of 150% based on starch weight. The solution was adjusted to pH 6.5 using concentrated hydrochloric acid (HCl) and mixing. Corn starch was added to the solution, mixed for 30 minutes, de-watered, and air dried. The air-dried starch was impregnated with STPP, transferred to an oven set at 152° C., and heat-treated for 45 minutes. The derivatized starch phosphate was added to water and generated a slurry having a final weight ratio of 1:1.5 of starch to water, providing a phosphorylated carbohydrate derivative.

Prophetic example: Carbohydrate derivatives having sulfonate anionic moieties are prepared using potato or dent corn as starch base materials. To prepare this derivatized carbohydrate, starch is slurried in water at a 1:1.25 starch to water ratio. Next, calcium hydroxide is added to achieve a final concentration of 2% calcium hydroxide based on starch weight. 1-choro-2-sulfopropionic acid (CSPA) is added to achieve a final concentration of 5%, based on amount of starch, while maintaining the pH at 11.2 using calcium hydroxide. The reaction is allowed to proceed for 1 hour at 40° C. The pH is lowered to 3.0 using HCl, then filter, wash with water, and dry. The starch sulfonate is reslurried into water to achieve a final weight ratio of 1:1.5 of starch to water, providing a sulfonated carbohydrate derivative.

### Example 2

Preparation of OSA modified starch

Native carbohydrate base or carbohydrate derivative was reacted with octenylsuccinic anhydride (OSA) under the following reaction conditions. A reaction slurry was prepared combining 100 parts starch (native or modified, as-is basis) to 125 parts water. The pH of the reaction mixture was raised to between 7.5-8.0 using 3% sodium hydroxide (NaOH). The desired amount of octenylsuccinate anhydride (1%-5% based on the weight of starch, anhydrous) was slowly added to the reaction mixture over 1.5 hrs. The reaction slurry was mixed for at least 4 hrs after complete addition of OSA. The pH was maintained at 7.5 using 3% NaOH. The reaction mixture was then brought to a pH of 5.0-6.0 using a 25% solution of hydrochloric acid (HCl). The starch was then recovered by vacuum filtration, washed with water, and dried in an oven overnight at 40-41° C.

### Example 3

Ionic Crosslinking of Starch with OSA

Starch samples described in Example 2 were treated with calcium for post modification crosslinking. A weighed amount of water (minus 10g for addition of NaOH) was prepared to obtain a final 30% solids slurry. Next was added 50g of anhydrous OSA modified starch (carboxylated derivative from Example 2) to the water. The pH of the mixture was adjusted using 1N NaOH (up to 10g) to achieve a final pH 8. Additional water was added so that the additional water and NaOH summed up to 10g. Next, calcium, in the form of calcium acetate monohydrate salt or other salt (such as those listed in Tables 1A and 1B), was dosed into the mixture and adjusted to pH 7, if necessary. The mixture was allowed to react for two hours. Product was filtered and dried. A detailed description of the generated samples can be found in Table 1. As a control, a sample was treated with aluminum in place of calcium.

When mixing the carbohydrate derivative esterified to octenyl succinic acid with a calcium source, the step used calcium at 4-20 times the stoichiometric equivalent to the anionic functional groups present on the carbohydrate. In order to quantify the anionic functional group present on the carbohydrate, one must calculate the % anionic functional groups (i.e. total carboxylate groups) present on the carbohydrate. This was done by, for example, mixing 5.0 g of starch with 25 mL 0.1N HCl and agitating the slurry for 1/2 hour. Next, vacuum filtering the slurry through a small Buchner funnel and wash the filter cake with 20 mL portions of purified water until the filtrate showed negative Cl” with AgNO3 or washing with sufficient quantity of purified water to ensure total removal of HCl. Next, transferring the washed filter cake (and filter paper if stuck to the starch cake) to a 600 mL beaker and slurry the starch with 100 mL of purified water. Add 200 mL of hot, purified water (approximately 95° C.), then standing the beaker in a boiling water bath and cook with stirring for 10 minutes. Removing the beaker from bath, adding 2-4 drops of phenolphthalein T.S., and while hot, titrating to the first permanent pink endpoint with 0.1N NaOH. Endpoint was defined as one which persisted for 15 seconds or longer. Calculation to determine % anionic functional groups was % COOH=[(mL NaOH) * (N NaOH) * (0.045)]/anhydrous sample weight * 100.

As shown in Table 1A, ranges of sodium hypochlorite and octenylsuccinic anhydride percentages were used upon a variety of starch sources to provide carboxylated OSA modified carbohydrate derivatives complexed with calcium.

TABLE 1A
Ionically crosslinked starch salts

| | | | | Type of |
|---|---|---|---|---|
| Sample | Starch | Sodium Hypochlorite | OSA | Polyvalent |
| ID | Base | (% active chlorine) | (%) | Metal |
| 1 | Corn | 0 | 2 | Aluminum |
| 2 | Waxy Corn | 0 | 3 | Calcium |
| 3 | Corn | 0.8 | 3 | Calcium |
| 4 | Potato | 0.8 | 2 | Calcium |
| 5 | Potato | 5.5 | 2 | Calcium |
| 6 | Potato | 0.8 | 3 | Calcium |
| 7 | Tapioca | 0.8 | 3 | Calcium |
| 8 | Corn | 0.0 | 2 | Calcium |
| 9 | Potato | 0.0 | 2 | Calcium |

Starch samples described in Example 2 (carboxylated OSA modified carbohydrate derivative) were treated with different polyvalent ions for post modification crosslinking, using the same procedure used for calcium treatment. As shown in Table 1B, carboxylated OSA modified carbohydrate derivatives were ionically crosslinked with iron, copper, zinc, and calcium.

TABLE 1B
Ionically crosslinked starch salts

| | | | | Type of |
|---|---|---|---|---|
| Sample | Starch | Sodium Hypochlorite | OSA | Polyvalent |
| ID | Base | (% active chlorine) | (%) | Metal |
| 10 | Corn | 0.8 | 3 | Iron |
| 11 | Corn | 0.8 | 3 | Copper |
| 12 | Corn | 0.8 | 3 | Zinc |
| 14 | Corn | 0.8 | 3 | Calcium |

Starch samples described in Example 1 (phosphorylated derivative) were treated to provide phosphorylated OSA modified carbohydrate derivative crosslinked with calcium. As shown in Table 1C, using the same procedure used for calcium treatment, except for using 30% STTP solution in place of sodium hypochlorite, was performed upon a starch base to provide phosphorylated OSA modified carbohydrate derivatives crosslinked to calcium.

TABLE 1C
Ionically crosslinked starch salts

| Sample | Starch | 30% STPP Solution | OSA | Type of |
|---|---|---|---|---|
| ID | Base | (% active STPP) | (%) | Polyvalent Metal |
| 13 | Corn | 4.6 | 3 | Calcium |

### Example 4

Water Repellency Testing

Samples described in Example 3 were tested for water repellency. This semi-quantitative method of hydrophobicity and water repellency encompassed very light sample agitation in a graduated centrifuge tube and observation for any resulting “wetted” material, which may settle to the bottom of the tube, was then measured and recorded.

Briefly, 5.00 grams of sample was added to 75 mL of purified water in a 100 mL Goetz type centrifuge tube. The tube was gently inverted 10 times, without shaking, and allowed to sit at room temperature for 1 hour. The tube was then inverted ten additional times, allowed to settle for 15 minutes, and the amount of material which settled to the bottom was read and recorded as average amount settled after 1.25 hours. A value less than 2 mL is considered water repellent and a value greater than 5 mL is considered as not water repellant. The results are shown in Table 2.

TABLE 2
Water repellency of ionically crosslinked derivatized starch salts

| Sample ID | Average Amount Settled after 1.25 hrs (mL) |
|---|---|
| 1 | 0 |
| 2 | 10 |
| 3 | 0.5 |
| 4 | 1 |
| 5 | 3 |
| 6 | 1.4 |
| 7 | 0.7 |
| 8 | 2.0 |
| 9 | 1.8 |
| 10 | 0 |
| 11 | 0 |
| 12 | 0 |
| 13 | 0.15 |
| 14 | 0.3 |

### Example 5

Characterization of Botanical Bases Octenylsuccinate modified starch derivative salts were analyzed for the properties of flowability, water repellency, and oil absorbency. The results are shown in Table 3. Additional samples were made and tested that contained 1-5% OSA. Such samples performed as expected.

Determination of free flow was made by examining the angle of repose from the dry powder. To determine the angle of repose, powder was first poured from an elevation onto a flat surface followed by determining the angle that the powder slope makes with respect to the horizontal surface (Hosokawa Powder Tester Model PT-X, Hosokawa Micron B.V.). This angle was qualified as the angle of repose. A smaller angle of repose generally implies enhanced free flow characteristics; that is, a composition having a smaller angle of repose is predicted to have greater flowability than a composition having a larger angle of repose.

Determination of water repellency was determined as described in Example 4.

Determination of % oil absorption was made by adding approximately 4 grams of sample into a centrifuge tube, then adding approximately 20 grams of oil into the tube. The tube was then vortexed on high for a few seconds, inverted a few times, and allowed to mix on a digital vortex mixer for 30 min. at speed of 1000. The slurry was centrifuged at 4750 rpm for 15 minutes for corn and tapioca 20 minutes for potato. The liquid was decanted into a tared 25 mL beaker and the weight recorded after 3.5 min (when no drops fell within 30 seconds). Oil absorption was measured as the difference of the total oil and decanted oil, divided by weight of sample [% Absorbed Oil=(liquid starting weight—decanted liquid)/sample weight * 100]. Generally, a composition having a higher oil absorption provides for enhanced performance in mitigating greasiness and providing preferred sensory attributes in many formulations, such as sunscreens, lotions, and ointments, compared to a composition having a higher oil absorption. In certain formulations, such as dry shampoos, a composition having the ability to absorb large amounts of oil is preferred.

TABLE 3
Physical characteristics of octenylsuccinate modified starches:
flowability, water repellency, and oil absorbency

| | | Average Amount | |
|---|---|---|---|
| | | Settled after 1.25 hr | |
| Sample ID | Angle of Repose (°) | (ml) | % Absorbed Oil |
| 2 | 37.4 | 10 | 66.00 |
| 3 | 24.2 | 0.5 | 55.4 |
| 6 | 30.7 | 1.4 | 57.8 |
| 7 | 24.3 | 0.7 | 48.1 |
| 8 | 35.0 | 2.0 | 49.7 |
| 9 | 28.7 | 1.8 | 38.5 |
| 10 | 27.9 | 0 | 59.36 |
| 11 | 28.1 | 0 | 58.52 |
| 12 | 28.4 | 0 | 60.27 |
| 13 | 29.7 | 0.15 | 56.78 |
| 14 | 28.6 | 0.3 | 54.4 |

### Example 6

Formulations of Sunscreens and Absorbency Measurements

Exemplary sunscreen formulations containing octenylsuccinate modified carbohydrate derivative salt (such as Sample No. 14) were prepared by the following steps using ingredients listed in Table 4A: added ingredient #1 into a main tank, then heat to 75-80° C. and add ingredients #2-3. Mixed until homogeneous. In a separate container, heated ingredients #4-10, then added into the main tank. Rinsed the oil phase in the separate container with ingredient #11, then added into main tank. Mixed for about 15-20 mins until homogeneous. Cooled the mixture to below 45° C., then added ingredients #12-13. Added ingredient #14, then mixed for about 10 min until homogeneous. Adjusted the pH to 5-6.5.

Table 4A provides a formulation for an organic version of sunscreen having a calculated sunscreen SPF 30, providing sunscreen formulations that are mineral-free or have reduced mineral content. Table 4B provides a formulation for a mineral-based version of sunscreen. Sunscreens containing octenylsuccinate modified carbohydrate derivative salt were prepared as mineral-based, mineral-reduced, or mineral-free formulations.

TABLE 4A
Formulation of sunscreen, SPF 30 (organic formulation)

| Ingredient | | | % in | |
|---|---|---|---|---|
| # | Trade name | Common Name | formula | Function |
| 1 | DI Water | Water | 44.55 | Carrier |
| 2 | Glystar A-31 | Sorbitol, Glycerin | 3.00 | Humectant |
| 3 | Crodesta F160-PW- | Sucrose Stearate | 1.00 | Thickener |
| | (JP) | | | |
| 4 | Dermofeel GSC | Glyceryl Stearate Citrate | 3.00 | Emulsifying Agent |
| 5 | Rita Cetyl Alcohol | Cetyl Alcohol | 5.00 | Emulsifying Agent |
| 6 | Grapeseed Oil | Vitis Vinifera (Grape) | 10.00 | Emollient |
| | | Seed Oil | | |
| 7 | Parsol 1789 | Avobenzone | 3.00 | Sunscreen Agent/UV |
| | | | | Absorber |
| 8 | Uvinul N 539 T | Octocrylene | 8.00 | Sunscreen Agent/UV |
| | | | | Absorber |
| 9 | Parsol HMS | Homosalate | 8.00 | Sunscreen Agent/UV |
| | | | | Absorber |
| 10 | Parsol EHS | Octisalate | 4.50 | Sunscreen Agent/UV |
| | | | | Absorber |
| 11 | DI Water | Water | 7.00 | Carrier |
| 12 | Microcare SBB | Benzoic acid, sorbic acid | 0.75 | Preservatives |
| | | (and) benzyl alcohol | | |
| 13 | Edeta BD | Disodium EDTA | 0.10 | Chelating Agent |
| 14 | Sample No. 14 | Modified carbohydrate of | 2.00 | Reduce Oiliness, |
| | | the invention | | produce soft |
| | | | | velvety skin feel |

Compositions described above were tested for skin absorbency by counting the number of rubs in circular rotations required for each composition to absorb into skin. Results are listed in Table 4B.

TABLE 5
Absorbency of sunscreen formulations made with octenylsuccinate
modified carbohydrate derivative salts

| Sample | Average # of Circular Rubs to Absorb in to Skin |
|---|---|
| 3 | 32 |
| 6 | 27 |
| 7 | 21 |
| 14 | 31 |

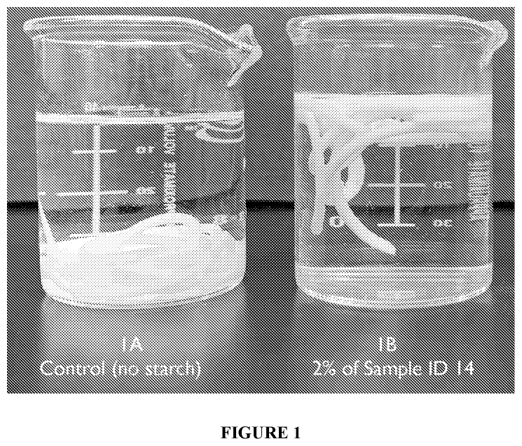

Sunscreen formulations were made according to Table 4A, either with or without a modified starch, and their water repellency was assessed as shown in FIG. 1. Approximately 2 ml of sunscreen SPF 30 formulation (FIG. 1B) or the control formulation made without starch (FIG. 1A) was added onto 50 ml of water. The sunscreen SPF 30 prepared with sample ID No. 14 floated on top of the water. In comparison, control sunscreen SPF 30 prepared without starch (line 14 of table 4A) settled to sit at the bottom of the container; it did not float in the water. It was observed that the sunscreen SPF 30 formulation prepared with sample ID No. 14 exhibited good water repellency.

An additional exemplary sunscreen was prepared by the following steps using ingredients from Table 4B: added ingredient #1 through #3 into a main tank, then heated to 70-75° C. Mixed until homogeneous. In a separate container (oil phase), heated ingredients #4 through #6, heat to 70-78° C. then added into the main tank. Rinsed the oil phase in the separate container with ingredient #7, then added into main tank. Mix for about 15-20 mins until homogeneous. Cooled the mixture to below 50° C., then add ingredients #8 through #12. Added ingredient #13, to adjust pH (5-6.5) then mixed until homogeneous.

Table 4B provided a formulation for a mineral-based version of sunscreen having a calculated sunscreen SPF 50. Table 4B provided a formulation for a mineral-based version of sunscreen. Sunscreens containing octenylsuccinate modified carbohydrate derivative salt were prepared as mineral-based, mineral-reduced, or mineral-free formulations.

TABLE 4B
Formulation of sunscreen, SPF 50 (mineral-based formulation)

| Ingredient | | | % in | |
|---|---|---|---|---|
| # | Trade name | Common Name | formula | Function |
| 1 | DI Water | Water | 51.98 | Carrier |
| 2 | Farmal Konjac | Glucomannan | 0.25 | Thickener |
| | 1310 | | | |
| 3 | Crodesta F160-PW- | Sucrose Stearate | 1.00 | Emulsifier Agent |
| | (JP) | | | |
| 4 | Dermofeel GSC | Glyceryl Stearate Citrate | 3.00 | Emulsifying Agent |
| 5 | Crodamol GTCC- | Caprylic/Capric Triglycerides | 5.00 | Emollient |
| | LQ-(SG) | | | |
| 6 | Grapeseed Oil | Vitis Vinifera (Grape) Seed | 10.00 | Emollient |
| | | Oil | | |
| 7 | DI Water | Water | 7.00 | Carrier |
| 8 | G-Block DT 200 | Titanium Dioxide (and) | 9.22 | Sunscreen Agent/UV |
| | CCT | Caprylic/Capric Triglyceride | | Absorber |
| | | (and) Isotearic Acid (and) | | |
| | | Alumina (and) | | |
| | | Polyhydroxystearic Acid | | |
| | | (and) Stearic Acid (and) | | |
| | | Polyglyceryl-3 Polyricinoleate | | |
| | | (and) Lecithin | | |
| 9 | G-Block DZ 370 | Zinc Oxide (and) | 9.30 | Sunscreen Agent/UV |
| | CCT | Caprylic/Capric Triglyceride | | Absorber |
| | | (and) Polyhydroxystearic | | |
| | | Acid (and) Polyglyceryl-3 | | |
| | | Polyricinoleate (and) | | |
| | | Isostearic Acid (and) Lecithin | | |
| 10 | Sample ID No. 14 | Modified carbohydrate of the | 2.00 | Reduce Oiliness, |
| | | invention | | produce soft velvety |
| | | | | skin feel |
| 11 | Microcare PHDG 2 | Phenoxyethanol, Caprylyl | 0.75 | Preservatives |
| | | Glycol, Decylene Glycol | | |
| 12 | Edeta BD | Disodium EDTA | 0.10 | Chelating Agent |
| 13 | Purac Hipure 90 | Lactic Acid | 0.40 | pH Adjuster |

### Example 7

Formulation of Body Powder

An exemplary body powder was prepared by the following steps using ingredients from Table 6: combined all ingredients (#1 through #4) and blended, for example in a ribbon blender, until uniform.

TABLE 6
Formulation of body powder

| Ingredient | | | | |
|---|---|---|---|---|
| # | Trade Name | Common Name | WT % | Function |
| 1 | Farmal CS 3757 | Zea Mays (Corn) Starch | 77.80 | Oil & Moisture Absorbent |
| | | | | Imparts soft velvety after-feel |
| 2 | Sample No. 14 | Modified carbohydrate | 21.00 | Oil & Moisture Absorbent |
| | | of the invention | | Imparts soft velvety after-feel |
| 3 | Calcium Phosphate | Tricalcium Phosphate | 1.00 | Flow Agent |
| | TB FCC PDR K | | | |
| 4 | Rose Powder | Fragrance | 0.20 | Aesthetics |
| | (ORC1206810z) | | | |
| | | TOTAL | 100.00 | |

### Example 8

Formulation of Dry Shampoo

Prophetic example: An exemplary dry shampoo containing a propellant is prepared by the following steps using ingredients from Table 7: added ingredient #1 in an appropriate tank. Slowly add ingredient #2 and mix until fully dispersed to create a concentrate phase. Fill cans with concentrated phase and add ingredient #3a or 3b.

TABLE 7
Formulation of dry shampoo containing propellant

| | Seq. # | Trade Name | Common Name | WT % | WT % | Function |
|---|---|---|---|---|---|---|
| Concentrate | 1 | SD Alcohol | Alcohol Denat. | 20.00 | 20.00 | Solvent |
| Phase | | 40-B | | | | |
| | 2 | Sample | Modified carbohydrate | 20.00 | 20.00 | Oil & Moisture |
| | | No. 14 | of the invention | | | Absorbent - Imparts |
| | | | | | | soft velvety after-feel |
| Propellant | 3a | A-46 | Isobutane (and) Propane | 60.00 | | Propellant |
| | 3b | 152a | Hydrofluorocarbon 152a | | 60.00 | Propellant |
| | | | TOTAL | 100.00 | 100.00 | |

An exemplary soft dry shampoo without a propellant was prepared by the following steps using ingredients from Table 8: added ingredients #1 through ingredient #4 (all components) in such order shown into a ribbon blender. Blended until completely homogenous.

TABLE 8
Formulation of soft dry shampoo without propellant

| Ingredient | | | | |
|---|---|---|---|---|
| # | Trade name | Common Name | % | Function |
| 1 | NativaCare 8600 | Oryza Sativa (Rice) | 67.00 | Oil & Moisture |
| | | Starch | | Absorbent, Imparts soft |
| | | | | velvety after-feel |
| 2 | Sample No. 14 | Modified carbohydrate | 30.00 | Oil & Moisture |
| | | of the invention | | Absorbent, Imparts soft |
| | | | | velvety after-feel |
| 3 | Calcium phosphate | Tricalcium phosphate | 1.00 | Anti-caking |
| | TB FCC PDR K | | | |
| 4 | Sodium bicarbonate | Sodium bicarbonate | 2.00 | Deodorant agent |
| | | TOTAL | 100.00 | |

### Example 9

Formulations of Color Cosmetic—Rosy Highlighter

Prophetic example: An exemplary rosy highlighter color cosmetic is prepared by the following steps using ingredients from Table 9A: combine ingredients #1 through ingredient #4 (all dry ingredients) and blend in a ribbon blender until uniform. Add Seq. #5 (Ritasol) & Seq. #6 (Cetiol C 5) to powder mixture and blend until uniform.

TABLE 9A
Formulation of rosy highlighter color cosmetic

| Ingredient | | | | |
|---|---|---|---|---|
| # | Trade Name | INCI Name | % | Function |
| 1 | Sample No. 14 | Modified carbohydrate of | 23.00 | Sensory |
| | | the invention | | |
| 2 | Winter Rose Mica | Mica, Titanium Dioxide, | 29.16 | Colorants/Pigments |
| | | iron Oxide & Tin Oxide | | |
| 3 | Ivory Lace | Mica, Titanium Dioxide, | 29.16 | Colorants/Pigments |
| | | iron Oxide & Boron Nitride | | |
| 4 | MicroCare PPD | Talc, Phenoxyethanol, | 1.00 | Preservatives |
| | | Decylene Glycol & | | |
| | | Hydrated Silica | | |
| 5 | Ritasol | Isopropyl Lanolate | 4.00 | Binding Agent |
| 6 | Cetiol C 5 | Coco-Caprylate | 13.68 | Emollient, Skin |
| | | | | Conditioning Agent |
| | | TOTAL | 100 | |

An exemplary rosy highlighter color cosmetic was prepared by the following steps using ingredients from Table 9: combined ingredients #1 through ingredient #4 (all dry ingredients) and blended in a ribbon blender until uniform. Added ingredient #5 (Ritasol) through ingredient #7 (Cetiol C 5) to powder mixture and blend until uniform. Press into tins.

TABLE 9B
Formulation of rosy highlighter color cosmetic

| Ingredient | | | | |
|---|---|---|---|---|
| # | Trade Name | INCI Name | % | Function |
| 1 | Sample No. 14 | Modified carbohydrate of | 31.40 | Sensory, Imparts soft |
| | | the invention | | velvety after-feel |
| 2 | Winter Rose | Mica, Titanium Dioxide, | 24.30 | Colorants/Pigments |
| | Mica | iron Oxide & Tin Oxide | | |
| 3 | Ivory Lace | Mica, Titanium Dioxide, | 24.30 | Colorants/Pigments |
| | | iron Oxide & Boron | | |
| | | Nitride | | |
| 4 | Geogard Ultra | Gluconolactone, Sodium | 1.00 | Preservatives |
| | | Benzoate | | |
| 5 | Ritasol | Isopropyl Lanolate | 4.00 | Binding Agent |
| 6 | Span 80-NV-LQ- | Sorbitan Oleate | 2.00 | Emulsifier |
| | (AP) | | | |
| 7 | Cetiol C 5 | Coco-Caprylate | 13.00 | Emollient, Skin |
| | | | | Conditioning Agent |
| | | TOTAL | 100.00 | |

### Example 10

Formulation of Color Cosmetic—Eyeshadow

Prophetic example: An exemplary eyeshadow color cosmetic is prepared by the following steps using ingredients from Table 10: combine ingredients #1 through ingredient #3 (all dry ingredients) and blend in a ribbon blender until uniform (note that in certain embodiments a single formulation includes any one of 2a, 2b, 2c, 2d or 2e). Next add ingredient #4 (Ritasol) & ingredient #5 (Eutanol G) to powder mixture and blend until uniform.

TABLE 10
Eyeshadow Formulation

| | | | Blue Green | Amethyst | Vibrant | Honey Mica | Smokey XXX | |
|---|---|---|---|---|---|---|---|---|
| | | | Pressed | Pressed | Pressed | Pressed | Pressed | |
| Ingr # | Trade Name | Common Name | Powder | Powder | Powder | Powder | Powder | Function |
| 1 | Sample | Modified carbohydrate | 37.00 | 37.00 | 37.00 | 37.00 | 37.00 | Sensory |
| | No. 14 | of the invention | | | | | | |
| 2a | Duocrome | Mica, Titanium Dioxide | 40.00 | | | | | Colorants/Pigments |
| | BY 226C | & Ferric Ferrocyanide | | | | | | |
| 2b | Gemtone | Mica, Titanium Dioxide, | | 40.00 | | | | Colorants/Pigments |
| | Amethyst | Ferric Ferrocyanide & | | | | | | |
| | G008 | Carmine | | | | | | |
| 2c | Duocrome | Mica, Titanium Dioxide | | | 40.00 | | | Colorants/Pigments |
| | Sparkle | & Carmine | | | | | | |
| | RY 224J | | | | | | | |
| 2d | Honey | Mica, Titanium Dioxide, | | | | 40.00 | | Colorants/Pigments |
| | Mica | iron Oxide & Tin Oxide | | | | | | |
| 2e | Smokey | Mica, Titanium Dioxide, | | | | | 40.00 | Colorants/Pigments |
| | XXX | iron Oxide & Tin Oxide | | | | | | |
| 3 | MicroCare | Talc, Phenoxyethanol, | 1.00 | 1.00 | 1.00 | 1.00 | 1.00 | Preservatives |
| | PPD | Decylene Glycol & | | | | | | |
| | | Hydrated Silica | | | | | | |
| 4 | Ritasol | Isopropyl Lanolate | 2.00 | 2.00 | 2.00 | 2.00 | 2.00 | Binding Agent |
| 5 | Eutanol G | Octyldodecanol | 20 | 20 | 20 | 20 | 20 | Emollient, Skin |
| | | | | | | | | Conditioning |
| | | | | | | | | Agent |
| | | TOTAL | 100 | 100 | 100 | 100 | 100 | |

### Example 11

Formulation for Ointment

An exemplary ointment was prepared by the following steps using ingredients from Table 11: combined ingredients #1 through ingredient #4 in a tank and mixed at 75-75° C. for 10 - 15 min. Ensured all waxes were melted and mixed until homogenous. Added ingredient #5 in the tank and mixed until completely dispersed. This was hot pour product. Filled the product at 60-65° C.

TABLE 11
Ointment Formulation

| Ingredient | | | | |
|---|---|---|---|---|
| # | Trade name | Common Name | % | Function |
| 1 | Snow White Pet USP, | Petrolatum | 50.00 | Skin Protectants (Ointment) |
| | Bulk | | | |
| 2 | Drakeol 35 min Oil, | Mineral Oil | 32.00 | Skin Conditioning Agent |
| | USP | | | |
| 3 | Ozokerite Wax Pastilles | Ozokerite | 3.00 | Viscosity Increasing Agent - |
| | | | | Nonaqueous |
| 4 | Paraffin Wax Pastilles | Paraffin | 5.00 | Viscosity Increasing Agent - |
| | | | | Nonaqueous |
| 5 | Sample No. 14 | Modified | 10.00 | Oil & Moisture Absorbent, |
| | | carbohydrate of | | Imparts soft velvety after-feel |
| | | the invention | | |
| | | TOTAL | 100.00 | |

### Example 12

Formulation for Anti-perspirant

Prophetic example: An exemplary anti-perspirant is prepared by the following steps using ingredients from Table 12: add ingredients #1 through ingredient #5 in a tank and mix at 75-80° C. for 10-15 min. Ensure all waxes are melted and mix until homogenous. Add ingredient #6 in and mix until completely dispersed. This is a hot pour product. Fill the product at 65-70° C.

TABLE 12
Anti-perspirant Formulation

| Ingredient | | | | |
|---|---|---|---|---|
| # | Trade name | Common Name | % | Function |
| 1 | Rita SA | Stearyl Alcohol | 15.00 | Emulsifier |
| 2 | Cremophor RH 40 | PEG-40 Hydrogenated | 4.00 | Skin Conditioning Agent |
| | | Castor Oil | | |
| 3 | Xiameter PMX | Cyclopentasiloxane, | 35.00 | Skin Conditioning Agent |
| | 345 | Cyclohexasiloxane | | |
| 4 | Cetiol C 5 | Coco-Caprylate | 21.00 | Emollient, Skin |
| | | | | Conditioning Agent |
| 5 | Drygen S | Aluminum Chlorohydrate | 20.00 | Anti-Perspirant |
| 6 | Sample No. 14 | Modified carbohydrate of | 5.00 | Oil & Moisture |
| | | the invention | | Absorbent Imparts soft |
| | | | | velvety after-feel |
| | | TOTAL | 100.00 | |

Prophetic example: An exemplary roll on anti-perspirant is prepared by the following steps using ingredients from Table 13: Add ingredients #1 through ingredient #6 in a tank and mix at 75-80° C. for 10-15 min. Ensure all waxes are melted and mix until homogenous. Add ingredient #7 in the tank and mix until completely dispersed.

TABLE 13
Roll on Anti-perspirant Formulation

| Ingredient | | | | |
|---|---|---|---|---|
| # | Trade name | Common Name | % | Function |
| 1 | DI Water | Water (Aqua) | 52.00 | Carrier |
| 2 | Emulgade NLB | Steareth-2, Ceteareth- | 5.00 | Emulsifier |
| | | 12, Stearyl Alcohol, | | |
| | | Ceteareth-20, Distearyl | | |
| | | Ether | | |
| 3 | Cremophor RH 40 | PEG-40 Hydrogenated | 4.00 | Skin Conditioning |
| | | Castor Oil | | Agent |
| 4 | Rita Coconut Oil 76 | Coconut Oil | 4.00 | Skin Conditioning |
| | | | | Agent |
| 5 | Cetiol C 5 | Coco-Caprylate | 10.00 | Emollient, Skin |
| | | | | Conditioning Agent |
| 6 | Drygen S | Aluminum | 20.00 | Anti-Perspirant |
| | | Chlorohydrate | | |
| 7 | Sample No. 14 | Modified carbohydrate | 5.00 | Oil & Moisture |
| | | of the invention | | Absorbent, Imparts |
| | | | | soft velvety after-feel |
| | | TOTAL | 100.00 | |

### Example 13

Formulation for Conditioner

An exemplary conditioner was prepared by the following steps using ingredients from Table 14: added ingredient #1 in the main tank. Premix ingredient #2-3 in a side tank and added in the main tank 70-75° C. for 10-15 min. Rinsed the pre-mix container with ingredient #4 and added in the main tank. Added ingredient #5 in the main tank. In a side tank (oil phase), mix ingredients #6 to #9. Ensured all waxes are melted and mix until homogenous at 70-75° C. Added Oil phase to the main tank and mix at 70-75° C. for 10-15 min. Rinsed oil phase container with ingredient #10 and added to the main tank. Added ingredient #11 and started to cool down. Below 50° C., added ingredients #12 through #15 and mixed until homogenous.

TABLE 14
Conditioner with Zinc Pyrithione Formulation

| Ingredient | | | % in | |
|---|---|---|---|---|
| # | Trade name | Common Name | formula | Function |
| 1 | DI Water | Water | 46.30 | Carrier |
| 2 | Glycerin USP - | Glycerin | 1.00 | Humectant |
| | 99.7% | | | |
| 3 | Pencare DP | Starch | 1.00 | Conditioning |
| | 1015 | Hydorxypropyltrimonium | | Agent |
| | | Chloride | | |
| 4 | DI Water | Water | 3.00 | Carrier |
| 5 | Sample No. 14 | Modified carbohydrate of the | 2.00 | Sensory |
| | | invention | | |
| 6 | Dermofeel | Glyceryl Stearate Citrate | 3.00 | Emulsifying |
| | GSC | | | Agent |
| 7 | Rita CA Cetyl | Cetyl Alcohol | 3.00 | Emulsifying |
| | Alcohol | | | Agent |
| 8 | Varisoft BTMS | Behentrimonium | 2.00 | Conditioning |
| | Pellets | Methosulfate, Cetearyl | | Agent & |
| | | Alcohol | | Emulsifier |
| 9 | Coconut Oil | Cocos Nucifera (Coconut) | 2.00 | Conditioning |
| | RDB (76) | Oil | | Agent |
| 10 | DI Water | Water | 5.00 | Carrier |
| 11 | DI Water | Water | 30.00 | Carrier |
| 12 | Zinc Omadine | Pyrithione Zinc | 1.02 | Anti-Dandruff |
| | 48% FPS | | | |
| 13 | Microcare ITO | Magnesium Nitrate, | 0.08 | Preservatives |
| | | Methylchloroisothiazolinone, | | |
| | | Magnesium Chloride (and) | | |
| | | Methylisothiazolinone | | |
| 14 | Microcare PE | Phenoxyethanol | 0.50 | Preservative |
| 15 | 25% Sodium | Sodium Hydroxide | 0.05 | pH Adjuster |
| | Hydroxide | | | |
| | | TOTAL | 100.00 | |

### Example 14

Formulation for Color Cosmetic—Rosy Loose Powder

An exemplary rosy highlighter color cosmetic was prepared by the following steps using ingredients from Table 15: combined ingredients #1 through ingredient #4 (all dry ingredients) and blended in a ribbon blender until uniform.

TABLE 15
Formulation of color cosmetic - rosy loose powder

| Ingredient | | | | |
|---|---|---|---|---|
| # | Trade Name | INCI Name | % | Function |
| 1 | Sample No. 14 | Modified carbohydrate of | 39.00 | Oil & Moisture |
| | | the invention | | Absorbent, Imparts |
| | | | | soft velvety after-feel |
| 2 | Winter Rose | Mica, Titanium Dioxide, | 30.00 | Colorants/Pigments |
| | Mica | iron Oxide & Tin Oxide | | |
| 3 | Ivory Lace | Mica, Titanium Dioxide, | 30.00 | Colorants/Pigments |
| | | iron Oxide & Boron | | |
| | | Nitride | | |
| 4 | Geogard Ultra | Gluconolactone, Sodium | 1.00 | Preservatives |
| | | Benzoate | | |
| | | TOTAL | 100.00 | |

1. A modified carbohydrate, wherein the modified carbohydrate is produced from a carbohydrate base being:
a) derivatized with one or more anionic moieties;
b) esterified with octenylsuccinic acid; and
c) complexed with a polyvalent cation.

2. The modified carbohydrate of claim 1, wherein the one or more anionic moieties is selected from the group consisting of carboxylate, sulfonate, phosphate, and mixtures thereof.

3. The modified carbohydrate of claim 1, wherein the carbohydrate base is selected from starch, cellulose and gum; and
optionally, wherein the carbohydrate base is starch sourced from the group consisting of corn, high amylose corn, waxy corn, potato, pea, rice, waxy rice, sago, tapioca, waxy tapioca, and mixtures thereof.

4. The modified carbohydrate of claim 1, wherein the polyvalent cation is selected from the group consisting of metal or alkaline earth metal ions; and
optionally, wherein the polyvalent cation is selected from the group consisting of calcium, zinc, copper, iron, and titanium.

5. The modified carbohydrate of claim 1, wherein the angle of repose of the modified carbohydrate is between about 20 to about 35.

6. The modified carbohydrate of claim 1, wherein the carbohydrate base is starch, the functional anionic moiety is carboxylate and the polyvalent cation is calcium.

7. The modified carbohydrate of claim 1, wherein the modified carbohydrate has a property selected from the following: free flowing, oil absorbing and water repellant, and mixtures thereof.

8. A personal care, food, or industrial composition comprising the modified carbohydrate of claim 1;
optionally, wherein the composition is a cosmetic, sunscreen, lotion, dry shampoo, anti-perspirant, deodorant, body powder, ointment, conditioner.

9. A method of using the composition of claim 8 as an ingredient in a food, cosmetic, dry shampoo, anti-perspirant, deodorant, ointment, sunscreen or industrial application.

10. Use of the composition of claim 8 in a food, cosmetic, dry shampoo, anti-perspirant, deodorant, ointment or sunscreen application.

11. A method of preparing a modified carbohydrate comprising:
a) treating a carbohydrate base to produce a carbohydrate derivative;
b) esterifying the carbohydrate derivative with octenylsuccinic anhydride to produce a carbohydrate derivative esterified with octenyl succinic acid; and
c) mixing the carbohydrate derivative esterified with octenyl succinic acid with a polyvalent cation.

12. The method of claim 11, wherein the polyvalent cation comprises calcium;
optionally, wherein the polyvalent cation is selected from calcium acetate or calcium chloride; and
optionally, wherein prior to mixing with the polyvalent cation, the esterified carbohydrate derivative is at a pH of about 8.

13. The method of claim 11, wherein the treating step uses sodium hypochlorite at a level of about 0.1 to about 17%, about 0.1 to about 15%, about 0.1 to about 13%, about 0.1 to about 10%, about 0.1 to about 5%, about 0.1 to about 3%, about 0.1 to about 1%, or about 0.4 to about 1.2% active chlorine based on the weight of the carbohydrate base;
optionally, wherein the treating step uses sodium hypochlorite to treat at levels of about 0.4 to about 1.2% active chlorine based on the weight of the carbohydrate base.

14. The method of claim 11, wherein the esterifying step uses about 1% to about 5% of octenylsuccinic anhydride based on weight of carbohydrate.

15. The method of claim 11, wherein the mixing step uses calcium at 4-20 times the stoichiometric equivalent to the anionic functional groups present on the carbohydrate.
